SEQUENCE LISTING (20 sequences)

SEQ ID NO: 1 - DNA, 1050 bp, Pediococcus acidilactici
  source 1..1050: mol_type = genomic DNA
atgtggtcaccttagaacggctagctcctaaaaggttaccccaccggctttgggtgttacaaactctcatggtgtgacgggcggtgtgtacaaggcccgggaacgtattcaccgcggcatgctgatccgcgattactagcgattccgacttcgtgtaggcgagttgcagcctacagtccgaactgagaatggttttaagagattagctaaacctcgcggtttcgcgactcgttgtaccatccattgtagcacgtgtgtagcccaggtcataaggggcatgatgatttgacgtcgtccccaccttcctccggtttgtcaccggcagtctcactagagtgcccaactgaatgctggcaactagtaataagggttgcgctcgttgcgggacttaacccaacatctcacgacacgagctgacgacaaccatgcaccacctgtcattctgtccccgaagggaacgcctaatctcttaggttggcagaagatgtcaagacctggtaaggttcttcgcgtagcttcgaattaaaccacatgctccaccgcttgtgcgggcccccgtcaattcttttgagtttcaaccttgcggtcgtactccccaggcggattacttaatgcgttagctgcagcactgaagggcggaaaccctccaacacttagtaatcatcgtttacggcatggactaccagggtatctaatcctgttcgctacccatgctttcgagcctcagcgtcagttacagaccagacagccgccttcgccactggtgttcttccatatatctacgcatttcaccgctacacatggagttccactgtcctcttctgcactcaagtctcccagtttccaatgcacttcttcggttgagccgaaagctttcacattagacttaaaagaccgcctgcgctcgctttacgcccaataaatccggataacgcttgccacctacgtattaccgcggctgctggcacgtagttagccgtggctttctggttaaataccgtcactgggtgacagttactctcacccacgttcttctttacacagagcttt

SEQ ID NO: 2 - DNA, 1042 bp, Pediococcus acidilactici
  source 1..1042: mol_type = genomic DNA
agagggtaatcggccacattgggactgagacacggcccagactcctacgggaggcagcagtagggaatcttccacaatggacgcaagtctgatggagcaacgccgcgtgagtgaagaagggtttcggatcgtaaagctctgttgttaaagaagaacgtgggtgagagtaactgttcacccagtgacggtatttaaccagaaagccacggctaactacgtgccagcagccgcggtaatacgtaggtggcaagcgttatccggatttattgggcgtaaagcgagcgcaggcggtcttttaagtctaatgtgaaagccttcggctcaaccgaagaagtgcattggaaactgggagacttgagtgcagaagaggacagtggaactccatgtgtagcggtgaaatgcgtagatatatggaagaacaccagtggcgaaggcggctgtctggctccggtaaactgacgctgaggctcgaaagcatgggtagcgaacaggattagataccctggtagtccatgccgtaaacgatgattactaagtgttggagggtttccgcccttcagtgctgcagctaacgcattaagtaatccgcctggggagtacgaccgcaaggttgaaactcaaaagaattgacgggggcccgcacaagcggtggagcatgtggtttaattcgaagctacgcgaagaaccttaccaggtcttgacatcttctgccaacctaagagattaggcgttcccttcggggacagaatgacaggtggtgcatggttgtcgtcagcctcgtgtcgtgagatgttgggttaagtcccgcaacgagcgcaacccttattactagttgccagcattcagttgggcactctagtgagactgccggtgacaaaccggaggaaggtggggacgacgtcaaatcatcatgccccttatgacctgggctacacacgtgctacaatggatggtacaacgagttgcgaaaccgcgaggtttagctaatctcttaaaaccattctcagttcggactgtaggctgcaactcgcctacacgaagt

SEQ ID NO: 3 - DNA, 1047 bp, Lactobacillus brevis
  source 1..1047: mol_type = genomic DNA
gctcgtaaaactctgttgttaaagaagaacacctttgagaagtaactgttcaagggttgacggtatttaaccagaaagccacggctaactacgtgccagcagccgcggtaatacgtaggtggcaagcgttgtccggatttattgggcgtaaagcgagcgcaggcggttttttaagtctgatgtgaaagccttcggcttaaccggagaagtgcatcggaaactgggagacttgagtgcagaagaggacagtggaactccatgtgtagcggtggaatgcgtagatatatggaagaacaccagtggcgaaggcggctgtctagtctgtaactgacgctgaggctcgaaagcatgggtagcgaacaggattagataccctgggtagtccatgccgtaaacgatgagtgctaagtgttggagggtttccgcccttcagtgctgcagctaacgcattaagcactccgcctggggagtacgaccgcaaggttgaaactcaaaaggaattgacgggggcccgcacaagcggtggagcatgtggtttaattcgaagctacgcgaagaaccttaccaggtcttgacatcttctgccaatcttagagataagacgttcccttcggggacagaatgacaggtggtgcatggttgtcgtcagctcgtgtcgtgagatgttgggttaagtcccgcaacgagcgcaacccttattatcagttgccagcattcagttgggcactctggtgagactgccggtgacaaaccgggaggaaggtggggatgacgtcaaatcatcatgccccttatgacctgggctacacacgtgctacaatggacggtacaacgagtcgcgaagtcgtgaggctaagctaatctcttaaagccgttctcagttcggattgtaggctgcaactcgcctacatgaagttggaatcgctagtaatcgcggatcagcatgccgcggtgaatacgttcccgggccttgtacacaccgcccgtcacaccatgagagtttgtaacacccaaagccggtgagataaccttcgggagtcagccgtc

SEQ ID NO: 4 - DNA, 1034 bp, Lactobacillus brevis
  source 1..1034: mol_type = genomic DNA
tatttaaccagaaagccacggctaactacgtgccagcagccgcggtaatacgtaggtggcaagcgttgtccggatttattgggcgtaaagcgagcgcaggcggttttttaagtctgatgtgaaagccttcggcttaaccggagaagtgcatcggaaactgggagacttgagtgcagaagaggacagtggaactccatgtgtagcggtggaatgcgtagatatatggaagaacaccagtggcgaaggcggctgtctagtctgtaactgacgctgaggctcgaaagcatgggtagcgaacaggattagataccctggtagtccatgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagctacgcgaagaaccttaccaggtcttgacatcttctgccaatcttagagataagacgttcccttcggggacagaatgacaggtggtgcatggttgtcgtcagctcgtgtcgtgagatgttgggttaagtcccgcaacgagcgcaacccttattatcagttgccagcattcagttgggcactctggtgagactgccggtgacaaaccggaggaaggtggggatgacgtcaaatcatcatgccccttatgacctgggctacacacgtgctacaatggacggtacaacgagtcgcgaagtcgtgaggctaagctaatctcttaaagccgttctcagttcggattgtaggctgcaactcgcctacatgaagttggaatcgctagtaatcgcggatcagcatgccgcggtgaatacgttcccgggccttgtacacaccgcccgtcacaccatgagagtttgtaacacccaaagccggtgagataaccttcgggagtcagccgtctaaggtgggacagatgattagggtgaagtcgtaacaaggtagccgtaggagaa

SEQ ID NO: 5 - DNA, 983 bp, Lactobacillus rhamnosus
  source 1..983: mol_type = genomic DNA
ccggatttattgggcgtaaagcgagcgcaggcggttttttaagtctgatgtgaaagccctcggcttaaccgaggaagtgcatcggaaactgggaaacttgagtacagaagaggacagtggaactccatgtgtagcggtgaaatgcgtagatatatggaagaacaccagtggcgaaggcggctgtctggtctgtaactgacgctgaggctcgaaagcatgggtagcgaacaggattagataccctggtagtccatgccgtaaacgatgaatgctaggtgttggagggtttccgcccttcagtgccgcagctaacgcattaagcattccgcctggggagtacgaccgcaaggttgaaactcaaaggaattgacgggggcccgcacaagcggtggagcatgtggtttaattcgaagcaacgcgaagaaccttaccaggtcttgacatcttttgatcacctgagagatcaggtttccccttcgggggcaaaatgacaggtggtgcatggttgtcgtcagctcgtgtcgtgagatgttgggttaagtcccgcaacgagcgcaacccttatgactagttgccagcatttagttgggcactctagtaagactgccggtgacaaaccggaggaaggtggggatgacgtcaaatcatcatgccccttatgacctgggctacacacgtgctacaatggatggtacaacgagttgcgagaccgcgaggtcaagctaatctcttaaagccattctcagttcggactgtaggctgcaactcgcctacacgaagtcggaatcgctagtaatcgcggatcagcacgccgcggtgaatacgttcccgggccttgtacacaccgcccgtcacaccatgagagtttgtaacacccgaagccggtggcgtaacccttttagggagcgagccgtctaaggtgggacaaatgattagggtgaagtcgtaacaaggtagccgtaggagaacctgcggctggatca

SEQ ID NO: 6 - DNA, 1031 bp, Lactobacillus rhamnosus
  source 1..1031: mol_type = genomic DNA
tccacaatggacgcaagtctgatggagcaacgccgcgtgagtgaagaaggctttcgggtcgtaaaactctgttgttggagaagaatggtcggcagagtaactgttgtcggcgtgacggtatccaaccagaaagccacggctaactacgtgccagcagccgcggtaatacgtaggtggcaagcgttatccggatttattgggcgtaaagcgagcgcaggcggttttttaagtctgatgtgaaagccctcggcttaaccgaggaagtgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagcatgggtagcgaacaggattagataccctggtagtccatgccgtaaacgatgaatgctaggtgttggagggtttccgcccttcagtgccgcagctaacgcattaagcattccgcctggggagtacgaccgcaaggttgaaactcaaaggaattgacgggggcccgcacaagcggtggagcatgtggtttaattcgaagcaacgcgaagaaccttaccaggtcttgacatcttttgatcacctgagagatcaggtttccccttcgggggcaaaatgacaggtggtgcatggttgtcgtcagctcgtgtcgtgagatgttgggttaagtcccgcaacgagcgcaacccttatgactagttgccagcatttagttgggcactctagtaagactgccggtgacaaaccggaggaaggtggggatgacgtcaaatcatcatgccccttatgacctgggctacacacgtgctacaatggatggtacaacgagttgcgagaccgcgaggtcaagctaatctcttaaagccattctcagttcggactgtangctgcaactcgcctacacgaagtcggaatcgctagtaatcgcggatcagcacgccgcggtgaatacgttcccgggccttgtacaca

SEQ ID NO: 7 - DNA, 1035 bp, Pediococcus acidilactici
  source 1..1035: mol_type = genomic DNA
aagaacgtgggtgagagtaactgttcacccagtgacggtatttaaccagaaagccacggctaactacgtgccagcagccgcggtaatacgtaggtggcaagcgttatccggatttattgggcgtaaagcgagcgcaggcggtcttttaagtctaatgtgaaagccttcggctcaaccgaagaagtgcattggaaactgggagacttgagtgcagaagaggacagtggaactccatgtgtagcggtgaaatgcgtagatatatggaagaacaccagtggcgaaggcggctgtctggtctgtaactgacgctgaggctcgaaagcatgggtagcgaacaggattagataccctggtagtccatgccgtaaacgatgattactaagtgttggagggtttccgcccttcagtgctgcagctaacgcattaagtaatccgcctggggagtacgaccgcaaggttgaaactcaaaagaattgacgggggcccgcacaagcggtggagcatgtggtttaattcgaagctacgcgaagaaccttaccaggtcttgacatcttctgccaacctaagagattaggcgttcccttcggggacagaatgacaggtggtgcatggttgtcgtcagctcgtgtcgtgagatgttgggttaagtcccgcaacgagcgcaacccttattactagttgccagcattcagttgggcactctagtgagactgccggtgacaaaccggaggaaggtggggacgacgtcaaatcatcatgccccttatgacctgggctacacacgtgctacaatggatggtacaacgagttgcgaaaccgcgaggtttagctaatctcttaaaaccattctcagttcggactgtaggctgcaactcgcctacacgaagtcggaatcgctagtaatcgcggatcagcatgccgcggtgaatacgttcccgggccttgtacacaccgcccgtcacaccatgagagtttgtaacacccaaagccggtggggtaaccttttaggagctagccgtctaaggtgaccagaat

SEQ ID NO: 8 - DNA, 1062 bp, Pediococcus acidilactici
  source 1..1062: mol_type = genomic DNA
gctcgtaaagctctgttgttaaagaagaacgtgggtgagagtaactgttcacccagtgacggtatttaaccagaaagccacggctaactacgtgccagcagccgcggtaatacgtaggtggcaagcgttatccggatttattgggcgtaaagcgagcgcaggcggtcttttaagtctaatgtgaaagccttcggctcaaccgaagaagtgcattggaaactgggagacttgagtgcagaagaggacagtggaactccatgtgtagcggtgaaatgcgtagatatatggaagaacaccagtggcgaaggcggctgtctggtctgtaactgacgctgaggctcgaaagcatgggtagcgaacaggattagataccctggtagtccatgccgtaaacgatgattactaagtgttggagggtttccgcccttcagtgctgcagctaacgcattaagtaatccgcctggggagtacgaccgcaaggttgaaactcaaaagaattgacgggggcccgcacaagcggtggagcatgtggtttaattcgaagctacgcgaagaaccttaccaggtcttgacatcttctgccaacctaagagattaggcgttcccttcggggacagaatgacaggtggtgcatggttgtcgtcagctcgtgtcgtgagatgttgggttaagtcccgcaacgagcgcaacccttattactagttgccagcattcagttgggcactctagtgagactgccggtgacaaaccggaggaaggtggggacgacgtcaaatcatcatgccccttatgacctgggctacacacgtgctacaatggatggtacaacgagtcgcgaaaccgcgaggtttagctaatctcttaaaaccattctcagttcggactgtaggctgcaactcgcctacacgaagtcggaatcgctagtaatcgcggatcagcatgccgcggtgaatacgttcccgggccttgtacacaccgcccgtcacaccatgagagtttgtaacacccaaagccggtggggtaaccttttaggagctagccgtctaaggtgatcacattggt

SEQ ID NO: 9 - DNA, 21 bp, synthetic construct
  source 1..21: mol_type = other DNA
gggcgataggagtccattcag

SEQ ID NO: 10 - DNA, 20 bp, synthetic construct
  source 1..20: mol_type = other DNA
acctggtggaactgggggta

SEQ ID NO: 11 - DNA, 26 bp, synthetic construct
  source 1..26: mol_type = other DNA
aaatattgtggatatcgggaggataa

SEQ ID NO: 12 - DNA, 22 bp, synthetic construct
  source 1..22: mol_type = other DNA
aggacggatggaaaaacctgta

SEQ ID NO: 13 - DNA, 21 bp, synthetic construct
  source 1..21: mol_type = other DNA
accacaacatgctgctccttt

SEQ ID NO: 14 - DNA, 22 bp, synthetic construct
  source 1..22: mol_type = other DNA
atcaacgtgcagccatatgtct

SEQ ID NO: 15 - DNA, 23 bp, synthetic construct
  source 1..23: mol_type = other DNA
tacgaggaaacgaaggtgaagtt

SEQ ID NO: 16 - DNA, 17 bp, synthetic construct
  source 1..17: mol_type = other DNA
ccgtagccgccagcttt

SEQ ID NO: 17 - DNA, 22 bp, synthetic construct
  source 1..22: mol_type = other DNA
aacgtcatgacccctcagaagt

SEQ ID NO: 18 - DNA, 22 bp, synthetic construct
  source 1..22: mol_type = other DNA
attcactgtgcctcaccctgta

SEQ ID NO: 19 - DNA, 23 bp, synthetic construct
  source 1..23: mol_type = other DNA
tagccttgtcagataaggaagga

SEQ ID NO: 20 - DNA, 22 bp, synthetic construct
  source 1..22: mol_type = other DNA
acagcttcacagtcaactttgt

(19) United States
(12) Patent Application Publication
(10) Pub. No.: US 2025/0325603 A1
(43) Pub. Date: Oct. 23, 2025

(54) PEDIOCOCCUS ACIDILACTICI TCI550, COMPOSITION HAVING THE SAME, AND METHOD FOR IMPROVING NEUROLOGICAL HEALTH AND/OR COGNITION BY USING THE SAME

(71) Applicant: TCI CO., LTD., Taipei (TW)

(72) Inventors: YUNG-HSIANG LIN, Taipei (TW); DI CHANG, Taipei (TW)

(21) Appl. No.: 19/064,718

(22) Filed: Feb. 27, 2025

Related U.S. Application Data
(60) Provisional application No. 63/636,114, filed on Apr. 19, 2024.

(51) Int. Cl. A61K 35/744; A61P 25/00; A61P 25/28

(52) U.S. Cl. CPC A61K 35/744; A61P 25/00; A61P 25/28

(57) ABSTRACT

Provided is a strain of Pediococcus acidilactici, which is P. acidilactici TCI550 deposited under an accession number of DSM34536. Provided is a method for improving neurological health and/or cognition by using P. acidilactici and/or metabolites thereof, including administering to a subject in need thereof a composition including an effective dose of P. acidilactici and/or metabolites thereof, wherein the P. acidilactici is P. acidilactici TCI550 deposited under an accession number of DSM34536.

### CROSS-REFERENCE TO RELATED APPLICATION

This application claims the benefit of U.S. provisional application Ser. No. 63/636,114, filed on Apr. 19, 2024. The entirety of the above-mentioned patent application is hereby incorporated by reference herein and made a part of the specification.

### REFERENCE OF AN ELECTRONIC SEQUENCE LISTING

The contents of the electronic sequence listing (P245884USI.xml; Size: 27,066 bytes; and Date of Creation: Feb. 20, 2025) is herein incorporated by reference in its entirety.

### BACKGROUND

### Technical Field

The instant disclosure relates to a strain of Pediococcus acidilactici and method thereof, and in particular, to a strain of P. acidilactici TC1550 and/or metabolites thereof, which can be used for preparing a composition for improving neurological health and/or cognition.

### Related Art

Probiotics are generally considered to be ingestible microorganisms that have positive benefits to a host (or referred to as a subject, such as an animal or human) after consumption. The nomenclature of probiotics originates from the Greek word “for life”, also referred to as “original health care strain.” Moreover, probiotics mainly refer to lactic acid bacteria and some yeast.

Generally, “lactic acid bacteria” refer to the general name of bacteria that can ferment carbohydrates to produce a large amount of lactic acid, and lactic acid bacteria are a rather complex flora. Among many strains of lactic acid bacteria, some strains of lactic acid bacteria are found to be good for the health of the subject. Therefore, these strains are regarded as probiotics. In other words, most probiotics are lactic acid bacteria, but only a minority of strains of lactic acid bacteria that have been proved to be good for the health of the subject can be referred to as probiotics.

By way of example, common strains of lactic acid bacteria that can be used as probiotics include Enterococcus, Lactobacillus, Bifidobacterium, Bacillus, etc., while strains of yeast that can be used as probiotics include Saccharomyces.

Lactobacillus is a group of gram-positive bacteria that has fermentability, is facultative anaerobic and does not produce spores. Lactobacillus is named for its ability to ferment the carbohydrates into the lactic acid, and can be used for making liquid yogurt, solid cheese, sauerkraut, beer, wine, pickles, pickled foods and other fermented foods.

### SUMMARY

In view of this, the instant disclosure provides a strain of P. acidilactici TCI550 and/or metabolites thereof, which have the functions of improving neurological health and/or cognition.

In some embodiments, a strain of P. acidilactici is provided. The P. acidilactici stain is P. acidilactici TC1550 deposited under an accession number of BCRC911167.

In some embodiments, a composition is provided. The composition includes P. acidilactici and a food additive, where the P. acidilactici is P. acidilactici TCI550 deposited under an accession number of DSM34536, and the food additive is at least one of a group consisting of: soybean milk powder, trehalose, indigestible maltodextrin, and sorbitol.

In some embodiments, use of P. acidilactici strain and/or metabolites thereof for preparing a composition for improving neurological health is provided. The P. acidilactici strain is P. acidilactici TC1550 deposited under an accession number of DSM34536.

In some embodiments, a method for improving neurological health is provided. The method for improving neurological health includes administering to a subject in need thereof a composition including an effective dose of P. acidilactici and/or metabolites thereof, wherein the P. acidilactici is P. acidilactici TC1550 deposited under an accession number of DSM34536.

In some embodiments, the improvement of neurological health is improving brain plasticity, calming nerves, or a combination thereof.

In some embodiments, the aforementioned composition for improving neurological health has a capability to reduce overall activity of sympathetic nerve cells.

In some embodiments, use of P. acidilactici strain and/or metabolites thereof for preparing a composition for improving cognition is provided. The P. acidilactici strain is P. acidilactici TCI550 deposited under an accession number of DSM34536.

In some embodiments, a method for improving cognition is provided. The method for improving cognition includes administering to a subject in need thereof a composition including an effective dose of P. acidilactici and/or metabolites thereof, wherein the P. acidilactici is P. acidilactici TCI550 deposited under an accession number of DSM34536.

In some embodiments, the aforementioned composition for improving cognition has a capability to improve responsiveness, concentration, memory, or any combination thereof.

In some embodiments, the aforementioned composition has a capability to increase expression levels of serotonin production-related gene, melatonin synthesis-related gene, depression resistance-related gene, or any combination thereof in a nerve cell.

In some embodiments, the aforementioned serotonin production-related gene is tryptophan hydroxylase (TPH) gene, dopa decarboxylase (DDC) gene, or a combination thereof.

In some embodiments, the melatonin synthesis-related gene is acetylserotonin O-methyltransferase (ASMT) gene, arylalkylamine acetyltransferase (AANAT) gene, or a combination thereof.

In some embodiments, the depression resistance-related gene is sirtuin 1 (SIRT1) gene, brain-derived neurotrophic factor gene, or a combination thereof.

In some embodiments, the aforementioned composition has a capability to promote secretion of γ-aminobutyric acid (GABA) by nerve cells.

In some embodiments, the P. acidilactici TCI550 has a capability to produce γ-aminobutyric acid and increase the level of the γ-aminobutyric acid in blood.

In some embodiments, the P. acidilactici TCI550 has a capability to increase the level of brain-derived neurotrophic factor (BDNF) in blood.

In some embodiments, the dosage of the aforementioned P. acidilactici TCI550 is 50 mg/day.

In summary, the P. acidilactici TCI550 and/or metabolites thereof according to any one of the embodiments are suitable for being prepared into a composition. The composition has the functions of improving neurological health and/or cognition. In some embodiments, the composition has at least one of the following abilities: producing the γ-aminobutyric acid, increasing the level of the γ-aminobutyric acid in blood, and increasing the level of the brain-derived neurotrophic factor in blood. In some embodiments, the composition has the ability to increase the expression level of at least one of the following genes: serotonin production-related gene, melatonin synthesis-related gene, and depression resistance-related gene in nerve cells. The serotonin production-related gene of the nerve cell may be, but is not limited to, tryptophan hydroxylase gene, dopa decarboxylase gene, or a combination thereof; the melatonin synthesis-related gene may be, but is not limited to, acetylserotonin O-methyltransferase (ASMT) gene, arylalkylamine acetyltransferase (AANAT) gene, or a combination thereof; and the depression resistance-related gene may be, but is not limited to, sirtuin 1 (SIRT1) gene, brain-derived neurotrophic factor gene, or a combination thereof. In some embodiments, the composition achieves the effect of improving neurological health through at least one of the following capabilities: reducing the overall activity of sympathetic nerve cells, improving brain plasticity, and calming nerves. In some embodiments, the composition achieves the effect of improving cognition through at least one of the following capabilities: improving responsiveness, concentration, and memory.

### BRIEF DESCRIPTION OF THE DRAWINGS

FIG. 1 is a bar chart showing detection data results of γ-aminobutyric acid (GABA) in various B. lacticum and P. acidilactici;

FIG. 2 is a bar chart showing detection results of the level of γ-aminobutyric acid (GABA) secreted by neuroblasts;

FIG. 3 is a bar chart showing detection results of the expression level of a brain-derived neurotrophic factor (BDNF) gene in neuroblasts;

FIG. 4 is a bar chart showing analysis experimental results of serotonin production-related gene, melatonin synthesis-related genes, and depression resistance-related gene;

FIG. 5 is a bar chart showing detection results of the average level of Y-aminobutyric acid (GABA) in the blood of subjects at week 0, week 2, and week 4;

FIG. 6 is a bar chart showing detection results of the average level of a brain-derived neurotrophic factor (BDNF) in the blood of subjects at week 0, week 2, and week 4;

FIG. 7 is a bar chart showing detection results of the activity of sympathetic nerve cells of subjects at week 0 and week 4;

FIG. 8 is a bar chart showing various evaluation results of sleep disorder severity of subjects at week 0 and week 4;

FIG. 9 is a bar chart showing evaluation results of the severity of various indexes of sleep quality of subjects at week 0 and week 4;

FIG. 10 is a bar chart showing results of cognitive disorder scores of subjects at week 0 and week 4;

FIG. 11 is a bar chart showing evaluation results of the severity of various indexes of cognitive function of subjects at week 0 and week 4;

FIG. 12 is a pie chart showing somatosensory questionnaire analysis of subjects for reducing the frequency of awakening at midnight after 4 weeks; and

FIG. 13 is a pie chart showing somatosensory questionnaire analysis of subjects for improving cognition after 4 weeks.

### DETAILED DESCRIPTION

P. acidilactici TCI550 is a strain of Pediococcus belonging to Lactobacteriaceae in Lactobacillies. The P. acidilactici TCI550 is spherical, and is usually present in pairs or in the form of tetrads. The P. acidilactici TCI550 is gram-positive bacterium, which can grow and reproduce in an anaerobic environment and is an anaerobic bacterium. In some embodiments, the P. acidilactici TCI550 can be directly isolated from human intestines.

The growth temperature of the P. acidilactici TCI550 is 25° C. to 45° C., and the preferred growth temperature is 30° C. to 37° C.

The P. acidilactici TCI550 has certain acid resistance and can survive in a low pH environment (e.g., yogurt and pickled food). Herein, the P. acidilactici TCI550 can survive in an environment with pH 4 to 7. In some embodiments, the P. acidilactici TCI550 is typically tolerant to an acidic environment with pH 4.0 or even less.

The P. acidilactici TCI550 was deposited at the Food Industry Research and Development Institute under an accession number of BCRC911167 on Jan. 11, 2023, and was deposited at the Leibniz Institute DSMZ-German Collection of Microorganisms and Cell Cultures (DSMZ; Address: Inhoffenstr. 7 B D-38124 Braunschweig), Germany, in accordance with the Budapest Treaty, under accession number of DSM34536 on Feb. 17, 2023.

In some embodiments, the P. acidilactici TCI550 and/or metabolites thereof have the ability to increase the level of γ-aminobutyric acid (GABA). The γ-aminobutyric acid can be secreted by the P. acidilactici TCI550. In addition, other cells can be induced by P. acidilactici TCI550 and/or metabolites thereof to secrete the γ-aminobutyric acid. In some embodiments, the P. acidilactici TCI550 has the ability to increase the level of the γ-aminobutyric acid in the blood of a subject. By way of example, when a subject takes the P. acidilactici TCI550, the P. acidilactici TCI550 that colonizes in the subject (such as the intestinal tract) will produce γ-aminobutyric acid, and the P. acidilactici TCI550 will also promote secretion of the γ-aminobutyric acid by nerve cells (such as neuroblasts), thereby increasing the level of the γ-aminobutyric acid in the blood of the subject. Moreover, when acting on a brain nerve cell, the γ-aminobutyric acid binds to a γ-aminobutyric acid subject to open a chloride ion channel on a cell membrane, and initiate inhibitory action potential to calm nerves. Therefore, the γ-aminobutyric acid contributes to relaxing the body and mind and aiding in sleep.

In some embodiments, the P. acidilactici TC1550 and/or metabolites thereof have the ability to increase the expression level of a brain-derived neurotrophic factor gene (referred to as BDNF gene) in nerve cells and increase the level of a brain-derived neurotrophic factor (BDNF) in the blood of a subject. By way of example, when a subject takes the P. acidilactici TCI550, the P. acidilactici TCI550 and metabolites thereof that colonize in the subject (such as the intestinal tract) will promote the expression of the BDNF gene in the nerve cells (e.g., neuroblasts) and secrete the brain-derived neurotrophic factor to increase the level of the brain-derived neurotrophic factor in the blood of the subject. The increase of the brain-derived neurotrophic factor contributes to neuroplasticity and strengthening brain. Therefore, the P. acidilactici TC1550 and/or metabolites thereof contribute to the neuroplasticity of the subject.

In some embodiments, the P. acidilactici TC1550 and/or metabolites thereof have the function of improving neurological health. The improvement of neurological health is multiple functions of improving brain plasticity, calming nerves and the like. By way of example, after the subject takes the P. acidilactici TCI550, the brain plasticity of the subject is improved and the nerves of the subject is calmed.

In some embodiments, the P. acidilactici TCI550 and/or metabolites thereof have the ability to reduce overall activity of sympathetic nerve cells of the subject. By way of example, after the subject takes the P. acidilactici TCI550 and/or metabolites thereof, the overall activity of the sympathetic nerve cells of the subject decreases. When the autonomic nerve of the subject is disordered, the sympathetic nerve of the subject will be in a state of excitement during sleep, so that the body and brain are unable to rest properly. Therefore, when the subject takes the P. acidilactici TCI550 and/or metabolites thereof, the overall activity of the sympathetic nerve of the subject decreases, which reduces the tension and excitement of the subject and helps the subject to fall asleep.

In some embodiments, the P. acidilactici TC1550 and/or metabolites thereof have the function of improving cognition. Specifically, the P. acidilactici TCI550 and/or metabolites thereof improve various cognitive index functions of the subject, such as responsiveness, concentration, and memory. By way of example, after the subject takes the P. acidilactici TC1550 and/or metabolites thereof, the responsiveness, concentration, and memory of the subject will be improved, and the cognitive ability of the subject is enhanced.

In some embodiments, the P. acidilactici TC1550 and/or metabolites thereof have the ability to increase expression levels of serotonin production-related gene, melatonin synthesis-related gene, depression resistance-related gene, or any combination thereof of nerve cell. The serotonin production-related gene is tryptophan hydroxylase (TPH) gene, dopa decarboxylase (DDC) gene, or a combination thereof; the melatonin synthesis-related gene is acetylserotonin O-methyltransferase (ASMT) gene, arylalkylamine acetyltransferase (AANAT) gene, or a combination thereof; and the depression resistance-related gene is sirtuin 1 (SIRT1) gene, brain-derived neurotrophic factor gene (BDNF), or a combination thereof.

As for the serotonin production-related gene, tryptophan hydroxylase edited by the TPH gene contributes to catalyzing the formation of serotonin, and the tryptophan hydroxylase is considered to play an important role in mood-related diseases (e.g., depression). Protein edited by the DDC gene contributes to catalyzing the formation of serotonin. Moreover, serotonin is an important related factor of learning and memory.

As for the melatonin synthesis-related gene, protein edited by the ASMT gene contributes to catalyzing the formation of melatonin. Protein edited by the AANAT gene can convert serotonin into N-acetylserotonin.

As for the depression resistance-related gene, the SIRT1 gene can demethylate histone to promote neural differentiation. Moreover, the activated SIRT1 gene contributes to reducing the inflammatory response of the brain nerves, which contributes to relieving depression, and the activated SIRT1 gene may play an important role in the pathological causes of severe depression. In addition, when the expression level of the SIRT1 gene is increased and the density of the nerve cells is regulated to increase density, relieving of depression is facilitated. As one of the important factors of an NTRK2 signaling pathway, the BDNF gene can promote the survival and differentiation of neuronal populations in peripheral and central nervous systems at a developmental stage, and has multiple effects of resisting depression, slowing down decline of the cognitive function, promoting the growth of new neurons and synapses, protecting existing neurons, maintaining long-term memory and the like.

In some embodiments, the metabolite of the P. acidilactici TCI550 is an active ingredient secreted by the P. acidilactici TCI550 to the extracellular space. In some embodiments, the metabolite of the P. acidilactici TCI550 may be TCI550 filtrate obtained after the broth of cultured P. acidilactici TCI550 is subjected to the steps of centrifuging, filtering to remove bacterial cells, and the like, and the filtrate may contain the active ingredients secreted by the P. acidilactici TCI550. By way of example, a bacterial broth obtained after culturing the P. acidilactici TCI550 overnight is centrifuged at 10,000×g for 10 min to obtain supernatant, and the supernatant is filtered through a 0.22 μm filter membrane to obtain the TCI550 filtrate, which contains the metabolites of the P. acidilactici TCI550 (including the active ingredients and the culture solution).

In some embodiments, the P. acidilactici TC1550 and/or metabolites thereof can be used for being prepared into a composition. The composition can be used for improving neurological health and/or cognition purposes.

In some embodiments, the aforementioned composition has various abilities to reduce overall activity of sympathetic nerve cells, increase expression levels of serotonin production-related genes (e.g., TPH gene, and DDC gene), melatonin synthesis-related genes (e.g., ASMT gene, and AANAT gene), depression resistance-related gene (e.g., SIRT1 gene, and BDNF gene), or a combination thereof, promote secretion of γ-aminobutyric acid by nerve cells, improve responsiveness, concentration, memory, or a combination thereof and the like.

The γ-aminobutyric acid contributes to promoting neuroplasticity and enhancing cognitive ability.

The brain-derived neurotrophic factor contributes to brain plasticity, which can mediate transport, is related to phosphorylation, and can enhance synaptic strength. Moreover, the brain-derived neurotrophic factor is directly related to brain learning and memory.

In some embodiments, the aforementioned composition includes a specific level of P. acidilactici TCI550 or metabolites thereof. By way of example, the composition has a specific dosage of P. acidilactici TCI550, and the effective dosage of the P. acidilactici TCI550 is 50 mg/day. Specifically, assuming that a portion of composition is administered once a day and the P. acidilactici TCI550 is dry powder, the composition at least includes 50 mg of P. acidilactici TCI550.

In some embodiments, the aforementioned subject is a human.

In some embodiments, the aforementioned composition may be a health-care product, a food product or a food additive for non-medical purposes. In other words, the health-care product, a food product or a food additive includes a specific dosage of P. acidilactici TCI550 or metabolites thereof.

In some embodiments, the aforementioned health-care product, food product or food additive may further include a food acceptable carrier widely used in a food manufacturing technology. For example, the pharmaceutically acceptable carrier may include one or more of the following reagents: a solvent, a buffer, an emulsifier, a suspending agent, a decomposer, a disintegrating agent, a dispersing agent, a binding agent, an excipient, a stabilizing agent, a chelating agent, a diluent, a gelling agent, a preservative, a wetting agent, a lubricant, an absorption delaying agent, a liposome and analogues. Selection and number regarding these reagents fall within the scope of the professional quality and routine technology well known to those skilled in the art.

In some embodiments, the food additive of the aforementioned health-care product, food product or food additive include a solvent selected from the group consisting of water, normal saline, phosphate buffered saline (PBS), and an aqueous solution containing alcohol.

In some embodiments, the food product may be, but is not limited to, beverages, fermented foods, bakery products, health foods for non-medical purposes, and dietary supplements.

In some embodiments, the composition may be present in the form of, but not limited to, powder, capsules, lozenges, granules, etc.

In some embodiments, the composition may only include the P. acidilactici TCI550. In some other embodiments, the composition includes the P. acidilactici TCI550 and at least one food additive, and the food additive is at least one of the group consisting of: soybean milk powder, trehalose, indigestible maltodextrin, and sorbitol.

In some embodiments, the composition is composed of the P. acidilactici TCI550, the soybean milk powder, the trehalose, the indigestible maltodextrin, and the sorbitol. In some examples, the composition is made by well mixing a P. acidilactici TCI550 bacterial broth, the soybean milk powder, the trehalose, the indigestible maltodextrin, and the sorbitol, then performing freeze drying and then grinding. By way of example, the P. acidilactici TCI550 is cultured at 37° C. for 24 h to obtain the P. acidilactici TCI550 bacterial broth, and 1.25% (w/w) of soybean milk powder, 1.25% (w/w) of trehalose, 0.5% (w/w) of indigestible maltodextrin, and 0.3% (w/w) of sorbitol are added into the P. acidilactici TCI550 bacterial broth according to the weight percentage of the bacterial broth, and then drying and grinding are performed after well mixing to obtain a mixture containing viable bacteria of the P. acidilactici TCI550, i.e., a bacterial powder composed of the viable bacteria of the P. acidilactici TCI550, the soybean milk powder, the trehalose, the indigestible maltodextrin, and the sorbitol.

In some embodiments, the composition may be a capsule containing bacterial powder of the viable bacteria of the P. acidilactici TCI550. By way of example, after the P. acidilactici TC1550 bacterial broth is freeze-dried and ground into the bacterial powder of the viable bacteria, an appropriate amount (such as 50 mg) of bacterial powder of the viable bacteria is taken and fills into the capsule to obtain a capsule containing the bacterial powder of the viable bacteria of the P. acidilactici TCI550. In some other embodiments, the composition may be a capsule containing the bacterial powder of the viable bacteria of the P. acidilactici TCI550 and other food additives. By way of example, after the bacterial powder of the viable bacteria is mixed with other food additives, an appropriate amount of the mixed bacterial powder is taken and fills into the capsule to obtain a capsule containing the viable bacteria of the P. acidilactici TCI550.

### Example 1: Strain Identification

Appropriate amounts of samples were taken from different sources (as shown in Table 1) and individually spread on a solid medium, and were cultured for 16 h in an anaerobic environment (i.e., the oxygen concentration in the culture environment was less than 1% by volume) at 37° C. until single colonies were formed. Here, the solid medium included 25 g/L of yeast peptone 902, 10 g/L of yeast peptone GLSF, 6 g/L of dipotassium hydrogen phosphate, 3 g/L of potassium dihydrogen phosphate, 1 g/L of cysteine, 5 g/L of sodium acetate, 0.1 g/L of magnesium sulfate, 1 g/L of polysorbate 80, 0.02 g/L of defoamer 30PS, 50 g/L of glucose, 20 g/L of 10N sodium hydroxide, 0.16 g/L of manganese gluconate, 1.7 g/L of citric acid, and 15 g/L of soybean peptide powder.

Next, multiple single colonies were picked from the solid Lactobacillus MRS medium, and strain identification was performed using 16S ribosomal gene (16SrDNA) sequences of the lactic acid bacteria. The 16SrDNA sequences of these single colonies were obtained by polymerase chain reaction (PCR), and after sequence alignment was performed between each of these gene sequences and the 16SrDNA sequences of other Lactobacillus strains by using the website of the National Center for Biotechnology Information (NCBI), possible strains of these single colonies can be inferred by using the similarity between the 16SrDNA sequences of these single colonies and other patterns or publicly available strains, and the identification results of the strains are shown in Table 1. The similarity between the single colony with a strain number TCI550 and a standard strain DSM20284 of the P. acidilactici (see Table 1 for “compared strains”) reached 99.33%, which indicates that the single colony with the strain number TCI550 was one of the strains of the P. acidilactici. Therefore, it was named P. acidilactici TCI550.

TABLE 1

| | | Sequence | | |
|---|---|---|---|---|
| | | number of | | |
| Strain | | 16SrDNA | | |
| Number | Isolation Source | sequence | Similarity | Compared strains |
| TCI550 | Human intestinal tracts | SEQ ID NO: 1 | 99.33% | P. acidilactici |
| | | | | (P. acidilactici DSM 20284) |
| LH426 | Baby intestinal tracts | SEQ ID NO: 2 | 99.42% | P. acidilactici |
| | | | | (P. acidilactici 8613) |
| LF417 | Pickles | SEQ ID NO: 3 | 99.62% | Lactobacillus brevis |
| | | | | (L. brevis WJY-3) |
| LF237 | Tempeh | SEQ ID NO: 4 | 99.90% | L. brevis |
| | | | | (L. brevis YSJ3) |
| LF358 | Fermented milt | SEQ ID NO: 5 | 99.90% | Lactobacillus rhamnosus |
| | | | | (L. rhamnosus VSI33) |
| LF165 | Fermented soybean milk | SEQ ID NO: 6 | 99.90% | L. rhamnosus |
| | | | | (L. rhamnosus KCC-51) |
| LF239 | Spicy pickled radish | SEQ ID NO: 7 | 99.81% | P. acidilactici |
| | | | | (P. acidilactici 8384) |
| LH349 | Human intestinal tracts | SEQ ID NO: 8 | 99.72% | P. acidilactici |
| | | | | (P. acidilactici 18-6B) |

Moreover, the P. acidilactici TCI550 is deposited at the Food Industry Research and Development Institute under accession number of BCRC911167, and is deposited at the Leibniz Institute DSMZ-German Collection of Microorganisms and Cell Cultures (DSMZ) under accession number of DSM34536.

### Example 2: Level of γ-aminobutyric Acid (GABA) Secreted by Multiple Lactic

### Acid Bacteria

8 lactic acid bacteria strains (i.e., P. acidilactici TCI550, P. acidilactici LH426, L. brevis LF417, L. brevis LF237, L. rhamnosus LF358, L. rhamnosus LF165, P. acidilactici LF239, and P. acidilactici LH349) in Table 1 were each inoculated into a liquid medium and cultured in an anaerobic environment (i.e., the oxygen concentration in the culture environment was less than 1% by volume) at 37° C. for 24 h to obtain a bacterial broth to be tested. Here, the liquid medium included 25 g/L of yeast peptone 902, 10 g/L of yeast peptone GLSF, 6 g/L of dipotassium hydrogen phosphate, 3 g/L of potassium dihydrogen phosphate, 1 g/L of cysteine, 5 g/L of sodium acetate, 0.1 g/L of magnesium sulfate, 1 g/L of polysorbate 80, 0.02 g/L of defoamer 30PS, 50 g/L of glucose, 20 g/L of 10N sodium hydroxide, 0.16 g/L of manganese gluconate, 1.7 g/L of citric acid, and 15 g/L of soybean peptide powder.

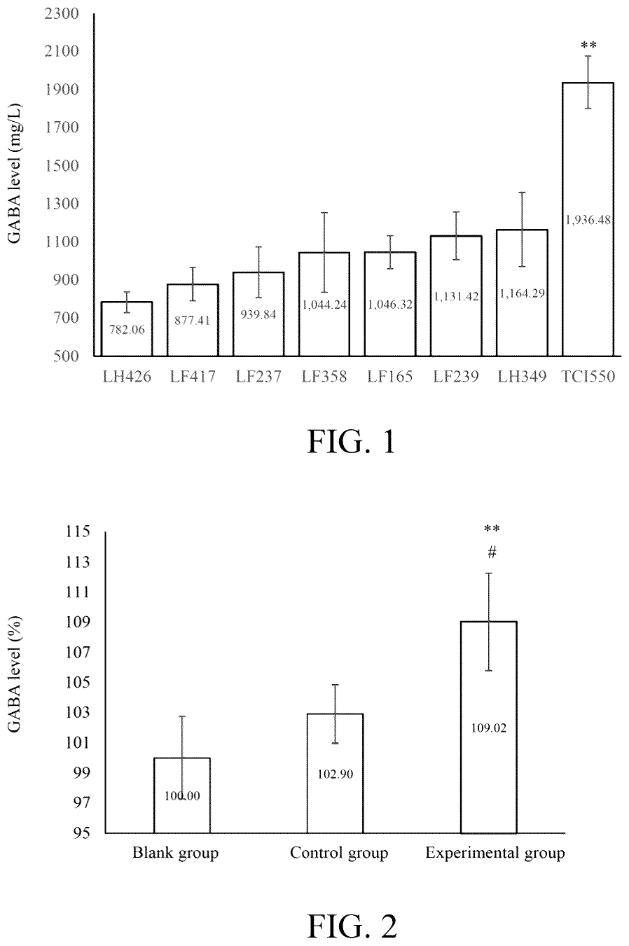

Next, the bacterial broth in each group were centrifuged at a speed of 5000 rpm for 15 min to isolate the bacterial cells and supernatant of lactic acid bacteria in each group. Then, the supernatant in each group was measured for the level of its contained γ-aminobutyric acid using a GABA level assay kit (brand: CLOUD-CLONE CORP; product number: CEA900Ge ELISA Kit for Gamma-Aminobutyric Acid (GABA)). The results are as shown in FIG. 1, where ** represents P<0.01, which shows the comparison between the P. acidilactici TCI550 and the P. acidilactici LH349.

Referring to FIG. 1. The GABA level of the supernatant of the P. acidilactici TCI550 was 1936.48 mg/L; the GABA level of the supernatant of the P. acidilactici LH426 was 782.06 mg/L; the GABA level of the supernatant of the L. brevis LF417 was 877.41 mg/L; the GABA level of the supernatant of L. brevis LF237 was 939.84 mg/L; the GABA level of the supernatant of the L. rhamnosus LF358 was 1044.24 mg/L; the GABA level of the supernatant of the L. rhamnosus LF165 was 1046.32 mg/L; the GABA level of the supernatant of the P. acidilactici LF239 was 1131.42 mg/L, and the GABA level of the supernatant of the P. acidilactici LH349 was 1164.29 mg/L. From this, it can be seen that the GABA level of the P. acidilactici TCI550 was nearly 2000 mg/L, which was much higher than that of a commercially available GABA product.

### Example 3: Analysis-HPLC Detection of Level of γ-aminobutyric Acid (GABA) Secreted by P. acidilactici TCI550 Itself

First, the P. acidilactici TCI550 identified in Example 1 was inoculated into a liquid medium and cultured in an anaerobic environment (i.e., the oxygen concentration in the culture environment was less than 1% by volume) at 37° C. for 24 h to obtain TCI550 bacterial broth. Here, the liquid medium included 25 g/L of yeast peptone 902, 10 g/L of yeast peptone GLSF, 6 g/L of dipotassium hydrogen phosphate, 3 g/L of potassium dihydrogen phosphate, 1 g/L of cysteine, 5 g/L of sodium acetate, 0.1 g/L of magnesium sulfate, 1 g/L of polysorbate 80, 0.02 g/L of defoamer 30PS, 50 g/L of glucose, 20 g/L of 10N sodium hydroxide, 0.16 g/L of manganese gluconate, 1.7 g/L of citric acid, and 15 g/L of soybean peptide powder.

Next, the TCI550 bacterial broth that has been cultured for 24 h was centrifuged at 10,000×g for 10 min to obtain TCI550 supernatant. Next, the TCI550 supernatant was filtered through a 0.22 μm filter membrane to obtain TCI550 filtrate for subsequent high performance liquid chromatography (HPLC) detection of the GABA level. Here, the HPLC analysis method was based on the research of scholars such as Somasundaram (Somasundaram, S., Tran, K. N. T., Ravikumar, S., & Hong, S. H. (2017). Biochemical Engineering Journal, 120, 1-6).

0.2 mL of TCI550 filtrate was taken and mixed with 0.1 mL of 0.5 mol/L of sodium bicarbonate (NaHCO₃, purchased from Sigma-Aldrich, USA) solution, and then 0.02 mL of 1% (v/v) of FDBN (purchased from Sigma-Aldrich, USA; diluted with acetonitrile) and 0.18 mL of ultrapure water were added, followed by well shaking. Next, a dark reaction was carried out in a water bath at 60° C. for 1 h to obtain a reaction solution. The reaction solution was cooled to room temperature, and 0.4 mL of 0.01 mol/L of potassium dihydrogen phosphate (KH₂PO₄, purchased from Sigma Aldrich, USA) solution was added, and the mixture was well shaken and stood for 15 min before centrifugation to obtain a solution to be tested.

10 μL of the solution to be tested was taken and analyzed by an HPLC instrument. A tubular column used by the HPLC instrument was an Agilent TC-C18 color spectrum column (4.6 mm×250 mm; purchased from the Agilent Technologies Taiwan Ltd.). A mobile phase A (solution A) used was a 0.05 mol/L of sodium acetate buffer solution (pH 5.7, the pH value was adjusted with glacial acetic acid with a volume fraction of 2%, and 30 mL/L of tetrahydrofuran was added), and a mobile phase B (solution B) was methanol (purchased from Sigma-Aldrich, USA). The flow rate used was 1 mL/min. The column temperature used was 28° C. The detection wavelength used was 360 nm.

The elution procedure used is shown in Table 2.

TABLE 2

| Time point | Solution |
|---|---|
| 0 min-20 min | 70% of solution A + 30% of solution B |
| 20 min-35 min | 50% of solution A + 50% of solution B |
| 35 min-60 min | 30% of solution A + 70% of solution B |
| 60 min-90 min | 0% of solution A + 100% of solution B |

Analysis results: The concentration of GABA in the TCI550 supernatant cultured for 24 h was 2131.10 ppm.

### Example 4: Detection of Level of γ-Aminobutyric Acid (GABA) Secreted by Nerve Cells Under Promotion by P. Acidilactici TCI550

Cell medium used was DMEM medium (Dulbecco's modified Eagle's medium; purchased from Gibco, Cat. No. 11965-092) supplemented with 10% of fetal bovine serum (FBS) (purchased from Gibco, Cat. No. 10437-028) and 1% of antibiotic (Anti-Anti; purchased from Gibco).

The nerve cell used was human nerve cell SHSY-5Y cell strain (ATCC® CRL-2266™; hereinafter referred to as SHSY-5Y cells).

The assay kit used was: GABA assay kit (brand: CLOUD-CLONE CORP; product number: CEA900Ge ELISA Kit for Gamma-Aminobutyric Acid (GABA)).

A sample to be tested used was the TCI550 filtrate prepared from the P. acidilactici TCI550 identified in Example 1. The P. acidilactici TCI550 was inoculated into a liquid medium and cultured in an anaerobic environment (i.e., the oxygen concentration in the culture environment was less than 1% by volume) at 37° C. for 24 h to obtain TCI550 bacterial broth. Next, the TCI550 bacterial broth that has been cultured for 24 h was centrifuged at 10,000×g for 10 min to obtain TCI550 supernatant, and the TCI550 supernatant was filtered through a 0.22 μm filter membrane to obtain TCI550 filtrate. Here, the liquid medium included 25 g/L of yeast peptone 902, 10 g/L of yeast peptone GLSF, 6 g/L of dipotassium hydrogen phosphate, 3 g/L of potassium dihydrogen phosphate, 1 g/L of cysteine, 5 g/L of sodium acetate, 0.1 g/L of magnesium sulfate, 1 g/L of polysorbate 80, 0.02 g/L of defoamer 30PS, 50 g/L of glucose, 20 g/L of 10N sodium hydroxide, 0.16 g/L of manganese gluconate, 1.7 g/L of citric acid, and 15 g/L of soybean peptide powder.

1×10⁵ SHSY-5Y cells were taken and inoculated into a 6-well culture plate containing 2 mL of cell medium, and cultured overnight at 37° C.

Next, the cell medium of the cell culture plate inoculated with the SHSY-5Y cells was replaced with an experimental medium and incubated at 37° C. for 6 h. The experimental medium varied according to the groups. The aforementioned SHSY-5Y cells were divided into two groups: a control group and an experimental group, where the experimental medium in the control group was cell medium, while the experimental medium in the experimental group was cell medium containing 0.25% of TCI550 filtrate.

Next, the cultured SHSY-5Y cells in each group were collected and treated with a lysis buffer. Then, the treated cells were placed at 12,000×g and centrifuged at 4° C. for 10 min to remove cell debris of the SHSY-5Y cells, and other insoluble substances. The supernatant after centrifugation was the sample to be tested.

The samples to be tested in the experimental group and the control group and the cell medium not cultured with SHSY-5Y cells (as the blank group) were each detected for its GABA level by the GABA assay kit and an ELISA reader. Moreover, the numerical value detected in the blank group was regarded as 100%, and was used as a comparison basis to convert the relative percentage of the experimental group and the control group. The analysis of the experimental results is shown in FIG. 2 (* represents P<0.05; ** represents P<0.01). The wavelength of the ELISA reader was set to 450 nm.

Referring to FIG. 2. The GABA level in the blank group was 100%, and the GABA level in the control group was 102.90%. There was no significant difference therebetween, which indicates no difference in GABA level. The GABA level in the experimental group was 109.02%. There was a significant difference between the experimental group and each of the blank group and the control group, which indicates that the metabolites of the P. acidilactici TCI550 (i.e., TCI550 filtrate) can promote secretion of GABA by the SHSY-5Y cells.

### Example 5: Analysis of Expression of Brain-Derived Neurotrophic Factor (BDNF) Gene

Here, gene detected was the brain-derived neurotrophic factor (BDNF) gene (GeneID: 627) (hereinafter referred to as BDNF gene). The BDNF gene was related to neuroplasticity, depression resistance, and cognitive ability enhancement.

Cell medium used was DMEM medium (Dulbecco's modified Eagle's medium; purchased from Gibco, Cat. No. 11965-092) supplemented with 10% of fetal bovine serum (FBS) (purchased from Gibco, Cat. No. 10437-028) and 1% of antibiotic (Anti-Anti; purchased from Gibco).

The nerve cell used was human nerve cell SHSY-5Y cell strain (ATCC® CRL-2266™; hereinafter referred to as SHSY-5Y cells).

A sample to be tested used was the TCI550 filtrate prepared from the P. acidilactici TCI550 identified in Example 1. The P. acidilactici TCI550 was inoculated into a liquid medium and cultured in an anaerobic environment (i.e., the oxygen concentration in the culture environment was less than 1% by volume) at 37° C. for 24 h to obtain TCI550 bacterial broth. Next, the TCI550 bacterial broth that has been cultured for 24 h was centrifuged at 10,000×g for 10 min to obtain TCI550 supernatant, and the TCI550 supernatant was filtered through a 0.22 μm filter membrane to obtain TCI550 filtrate. Here, the liquid medium included 25 g/L of yeast peptone 902, 10 g/L of yeast peptone GLSF, 6 g/L of dipotassium hydrogen phosphate, 3 g/L of potassium dihydrogen phosphate, 1 g/L of cysteine, 5 g/L of sodium acetate, 0.1 g/L of magnesium sulfate, 1 g/L of polysorbate 80, 0.02 g/L of defoamer 30PS, 50 g/L of glucose, 20 g/L of 10N sodium hydroxide, 0.16 g/L of manganese gluconate, 1.7 g/L of citric acid, and 15 g/L of soybean peptide powder.

1×10⁵ SHSY-5Y cells were taken and inoculated into containing 2 mL of cell medium, and cultured overnight at 37° C.

Next, the cells were divided into three groups: a blank group, a control group and an experimental group. Pure cell medium (without cultured cells) was used in the blank group, and the cultured SHSY-5Y cells were divided into a control group and an experimental group, and the cell media in the two groups were each replaced with an experimental medium, and cultured at 37° C. for 6 h. Herein, the experimental medium in the control group was cell medium, while the experimental medium in the experimental group was cell medium containing 0.25% of TCI550 filtrate.

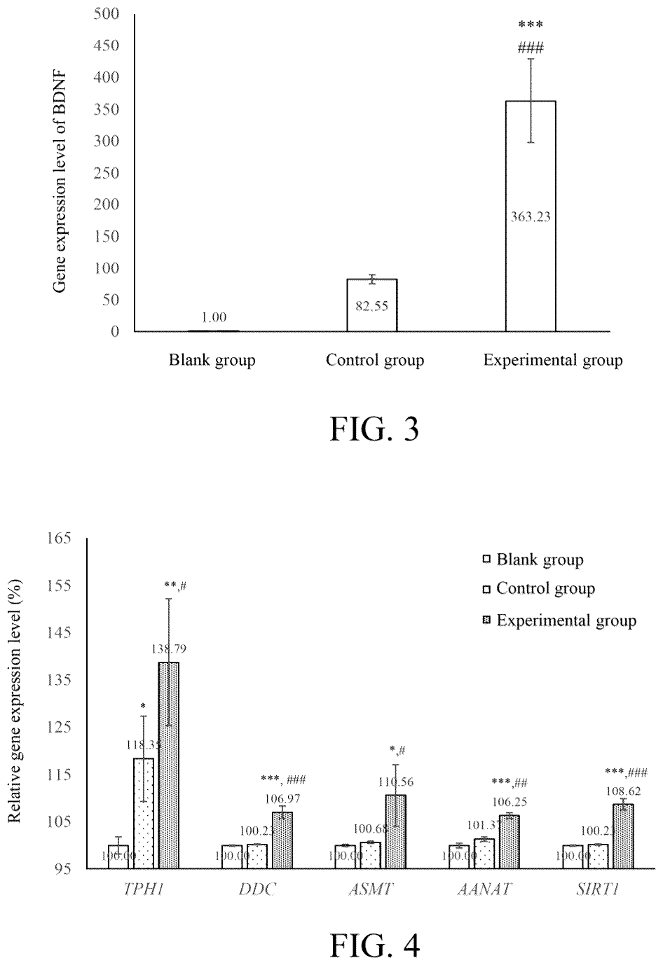

The SHSY-5Y cells in each group were collected, and RNA in each group was extracted using an RNA extraction reagent kit (purchased from Geneaid, Taiwan, Lot No. FC24015-G). Next, 2000 ng of RNA was taken from each group as a template, and was reverse transcribed into corresponding cDNA using SuperScript® III reverse transcriptase (purchased from Invitrogene, USA, No. 18080-051). A quantitative real-time reverse transcription polymerase chain reaction was then carried out on cDNA in each group by ABI StepOnePlus™ Real-Time PCR system (Thermo Fisher Scientific, USA), KAPA SYBR FAST (purchased from Sigma, USA, No. 38220000000), and primers in Table 3 (SEQ ID NO: 9 to SEQ ID NO: 10) to observe the expression level of the BDNF gene in the SHSY-5Y cells. Instrument set conditions for the quantitative real-time reverse transcription polymerase chain reaction were as follows: the reaction was carried out at 95° C. for 20 s, followed by the reaction at 95° C. for 3 s, and the reaction at 60° C. for 30 s, and the reactions were repeated for 40 cycles, and gene relative quantification was performed using a SCORE method, as shown in FIG. 3. Here, the quantitative real-time reverse transcription polymerase chain reaction by the cDNA can indirectly quantify the mRNA expression level of the gene, thereby inferring the expression level of protein encoded by the gene. Moreover, the gene expression level of BDNF in FIG. 3 is presented in relative magnification, where the expression level of the blank group is regarded as 1, and serves as a basis for calculating the gene expression levels of the control group and the experimental group.

TABLE 3

| | Primer | Sequence | | Primer |
|---|---|---|---|---|
| Target gene | name | number | Sequence | length |
| BDNF | BDNF_F | SEQ ID | GGGCGATAGGAGTCCATTCAG | 21 |
| | | NO: 9 | | |
| | BDNF_R | SEQ ID | ACCTGGTGGAACTGGGGGTA | 20 |
| | | NO: 10 | | |

In Table 3, F is a forward prime and R is a reverse prime.

Referring to FIG. 3. The relative expression level of the BDNF gene in the blank group was 1.00. Compared with that in the blank group, the expression level of BDNF gene in the control group was 82.55, and the expression level of BDNF gene in the experimental group was 363.23. From this, it can be seen that the expression level of the BDNF gene in the experimental group was significantly increased, which was about 360 folds higher than that in the blank group and 4 folds higher than that in the control group, which indicates that the metabolite of the P. acidilactici TCI550 (i.e., TCI550 filtrate) can effectively promote the expression of the BDNF gene in the nerve cells, and increase the expression level of BDNF. In other words, when the subject takes the P. acidilactici TCI550, the expression level of the BDNF gene can be increased, which in turn contributes to neuroplasticity and strengthening brain.

### Example 6: Analysis of Expression Levels of Serotonin Production-Related Gene, Melatonin Synthesis-Related Gene, and Depression Resistance-Related Gene

Here, the serotonin production-related gene detected was tryptophan hydroxylase 1 (TPH1) gene (Gene ID: 7166) (hereinafter referred to as TPH1 gene) and Dopa decarboxylase (DDC) gene (Gene ID: 1644) (hereinafter referred to as DDC gene). The melatonin synthesis-related gene detected was acetylserotonin O-methyltransferase (ASMT) gene (Gene ID: 438) (hereinafter referred to as ASMT gene) and arylalkylamine acetyltransferase (AANAT) gene (Gene ID: 15) (hereinafter referred to as AANAT gene). The depression resistance-related gene detected was Sirtuin (Sirtuin 1, SIRT1) gene (Gene ID: 23411) (hereinafter referred to as SIRT1 gene).

Cell medium used was DMEM medium (Dulbecco's modified Eagle's medium; purchased from Gibco, Cat. No. 11965-092) supplemented with 10% of fetal bovine serum (FBS) (purchased from Gibco, Cat. No. 10437-028) and 1% of antibiotic (Anti-Anti; purchased from Gibco).

The nerve cell used was human nerve cell SHSY-5Y cell strain (ATCCR CRL-2266™; hereinafter referred to as SHSY-5Y cells).

A sample to be tested used was the TCI550 filtrate prepared from the P. acidilactici TCI550 identified in Example 1. The P. acidilactici TCI550 was inoculated into a liquid medium and cultured in an anaerobic environment (i.e., the oxygen concentration in the culture environment was less than 1% by volume) at 37° C. for 24 h to obtain TCI550 bacterial broth. Next, the TCI550 bacterial broth that has been cultured for 24 h was centrifuged at 10,000×g for 10 min to obtain TCI550 supernatant, and the TCI550 supernatant was filtered through a 0.22 μm filter membrane to obtain TCI550 filtrate. Here, the liquid medium included 25 g/L of yeast peptone 902, 10 g/L of yeast peptone GLSF, 6 g/L of dipotassium hydrogen phosphate, 3 g/L of potassium dihydrogen phosphate, 1 g/L of cysteine, 5 g/L of sodium acetate, 0.1 g/L of magnesium sulfate, 1 g/L of polysorbate 80, 0.02 g/L of defoamer 30PS, 50 g/L of glucose, 20 g/L of 10N sodium hydroxide, 0.16 g/L of manganese gluconate, 1.7 g/L of citric acid, and 15 g/L of soybean peptide powder.

1×10⁵ SHSY-5Y cells were taken and inoculated into containing 2 mL of cell medium, and cultured overnight at 37° C.

Next, the cells were divided into three groups: a blank group, a control group and an experimental group. A pure cell medium (without cultured cells) was used in the blank group, and the cultured SHSY-5Y cells were divided into a control group and an experimental group, and the cell media in the two groups were each replaced with an experimental medium, and cultured at 37° C. for 6 h. Here, the experimental medium in the control group was cell medium, while the experimental medium in the experimental group was cell medium containing 0.25% of TCI550 filtrate.

The SHSY-5Y cells in each group were collected, and RNA in each group was extracted using an RNA extraction reagent kit (purchased from Geneaid, Taiwan, Lot No. FC24015-G). Next, 2000 ng of RNA was taken from each group as a template, and was reverse transcribed into corresponding cDNA using SuperScript® III reverse transcriptase (purchased from Invitrogene, USA, No. 18080-051). A quantitative real-time reverse transcription polymerase chain reaction was then carried out on cDNA in each group by ABI StepOnePlus™ Real-Time PCR system (Thermo Fisher Scientific, USA), KAPA SYBR FAST (purchased from Sigma, USA, No. 38220000000), and primers in Table 4 (SEQ ID NO:11 to SEQ ID NO:20) to observe the expression levels of various genes in the SHSY-5Y cells. Instrument set conditions for the quantitative real-time reverse transcription polymerase chain reaction were as follows: the reaction was carried out at 95° C. for 20 s, followed by the reaction at 95° C. for 3 s, and the reaction at 60° C. for 30 s, and the reactions were repeated for 40 cycles, and gene relative quantification was performed using a SCORE method, as shown in FIG. 4. Here, the quantitative real-time reverse transcription polymerase chain reaction by the cDNA can indirectly quantify the mRNA expression level of the gene, thereby inferring the expression level of protein encoded by the gene. Moreover, the gene expression levels of the serotonin production-related gene, the melatonin synthesis-related gene, and the depression resistance-related gene in FIG. 4 are presented in relative percentage, where the expression level of the blank group is regarded as 100%, and serves as a basis for calculating the gene expression levels of the control group and the experimental group.

TABLE 4

| | | Sequence | | Primer |
|---|---|---|---|---|
| Target gene | Primer name | number | Sequence | length |
| [Serotonin] | TPH1_F | SEQ ID | AAATATTGTGGATATCGGGAGGATAA | 26 |
| TPH1 | | NO: 11 | | |
| | TPH1_R | SEQ ID | AGGACGGATGGAAAAACCTGTA | 22 |
| | | NO: 12 | | |
| [Serotonin] | DDC_F | SEQ ID | ACCACAACATGCTGCTCCTTT | 21 |
| DDC | | NO: 13 | | |
| | DDC_R | SEQ ID | ATCAACGTGCAGCCATATGTCT | 22 |
| | | NO: 14 | | |
| [Melatonin] | ASMT_F | SEQ ID | TACGAGGAAACGAAGGTGAAGTT | 23 |
| ASMT | | NO: 15 | | |
| | ASMT_R | SEQ ID | CCGTAGCCGCCAGCTTT | 17 |
| | | NO: 16 | | |
| [Melatonin] | AANAT_F | SEQ ID | AACGTCATGACCCCTCAGAAGT | 22 |
| AANAT | | NO: 17 | | |
| | AANAT_R | SEQ ID | ATTCACTGTGCCTCACCCTGTA | 22 |
| | | NO: 18 | | |
| [Depression | SIRT1_F | SEQ ID | TAGCCTTGTCAGATAAGGAAGGA | 23 |
| resistance] | | NO: 19 | | |
| SIRT1 | SIRT1_R | SEQ ID | ACAGCTTCACAGTCAACTTTGT | 22 |
| | | NO: 20 | | |

In Table 4, F is a forward prime and R is a reverse prime.

Referring to FIG. 4. The expression levels of the TPH1 gene, the DDC gene, the ASMT gene, the AANAT gene, and the SIRT1 gene in the blank group were all 100%. Compared with that in the blank group, the expression level of the TPH1 gene in the control group was 118.35%, the expression level of the DDC gene was 100.23%, the expression level of ASMT gene was 100.68%, the expression level of the AANAT gene was 101.37%, and the expression level of the SIRT1 gene was 100.23%, which indicates that the SHSY-5Y cells are not affected by the pure cell medium. The expression level of the TPH1 gene in the experimental group was 138.79%, the expression level of the DDC gene was 106.97%, the expression level of the ASMT gene was 110.56%, the expression level of the AANAT gene was 106.25%, and the expression level of the SIRT1 gene was 108.62%. From this, it can be seen that the expression levels of various genes in the experimental group were significantly increased, which indicates that the metabolite of the P. acidilactici TCI550 (i.e., TCI550 filtrate) can effectively promote the expression of the serotonin production-related gene (such as the TPH1 gene and the DDC gene), the melatonin synthesis-related gene (such as the ASMT gene and the AANAT gene), and the depression resistance-related gene (such as the SIRT1 gene) in the nerve cells, and increase the expression level of protein corresponding to each gene. In other words, when the subject takes the P. acidilactici TCI550, the expression levels of the TPH1 gene, the DDC gene, the ASMT gene, the AANAT gene, and the SIRT1 gene are increased, which in turns contributes to various effects of improving neurological health, improving cognition, depression resistance and the like.

### Example 7: Human Body Test

Test method: The experiment was carried out with a viable bacteria capsule prepared from the P. acidilactici TC1550 identified in Example 1 (hereinafter referred to as a P. acidilactici TCI550 viable bacteria capsule), and the P. acidilactici TCI550 viable bacteria capsule contained 50 mg of bacterial powder. 10 subjects took one P. acidilactici TCI550 viable bacteria capsule after dinner/before bedtime every day for 4 consecutive weeks, and blood and sleep detection, and a somatosensory questionnaire survey were conducted before taking (week 0), 2 weeks after taking (week 2), and 4 weeks after taking (week 4). 10 subjects were aged 20 and above, who felt that their sleep quality was poor (for example, difficulty falling asleep, frequent awakening or early awakening). The bacterial powder was prepared by adding 1.25% (w/w) of soybean milk powder, 1.25% (w/w) of trehalose, 0.5% (w/w) of indigestible maltodextrin, and 0.3% (w/w) of sorbitol into the P. acidilactici TCI550 bacterial broth (i.e., the bacterial broth to be tested in Example 2) prepared in Example 2 according to the weight percentage of the bacterial broth, well mixing, freeze drying and grinding.

Test items: Blood detection-detection of GABA and BDNF levels in blood; autonomic nerve detection-sympathetic nerve cell activity (LF) detection; Somatosensory questionnaire survey-sleep disorder and cognitive disorder.

### 7-1. Blood Detection

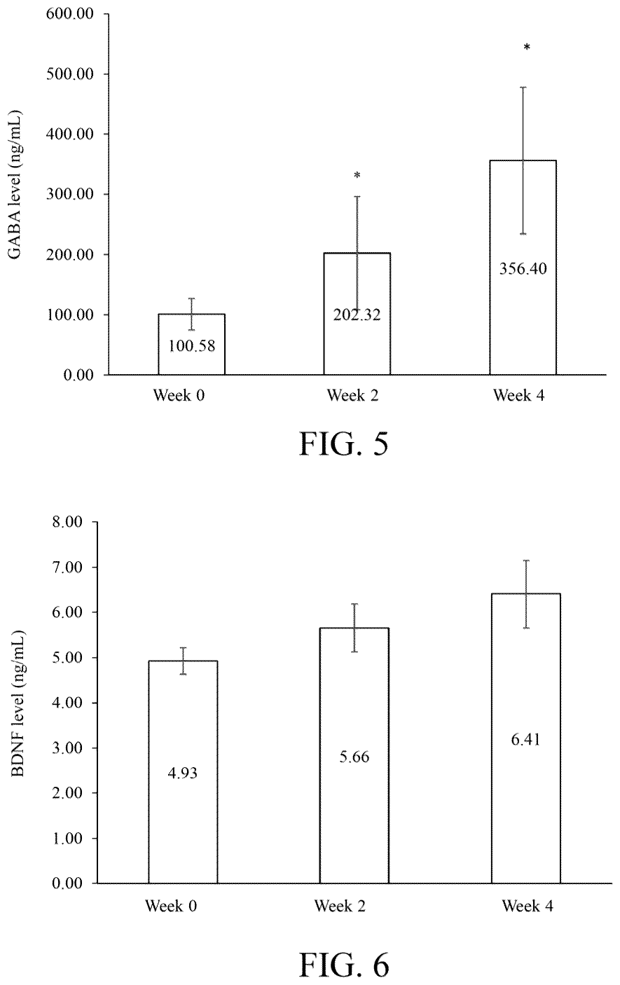

Blood detection method: Blood samples were collected from 10 subjects for detection before taking (i.e., week 0), 2 weeks after taking (week 2), and 4 weeks after taking (i.e., week 2), and the TCI Gene Inc. was entrusted to detect the level of the Gamma-aminobutyric acid (GABA) (as shown in FIG. 5) and the level of the brain-derived neurotrophic factor (BDNF) (as shown in FIG. 6) in the blood of the subjects.

Referring to FIG. 5. Before taking (i.e., week 0), the average GABA level of the blood of 10 subjects was 100.58 ng/mL. After taking for 2 consecutive weeks (i.e., week 2), the average GABA level of the blood of the 10 subjects was 202.32 ng/ml, which was increased by 101.1% compared to that at week 0. After taking for 4 consecutive weeks (i.e., week 4), the average GABA level of the blood of the 10 subjects was 356.40 ng/ml, which was increased by 254.3% compared to that at week 0. In other words, taking the P. acidilactici TCI550 viable bacteria capsule for 4 consecutive weeks can increase the GABA level in vivo. Moreover, more than half of the 10 subjects were improved, with the improvement rate reaching 90% (i.e., 9 out of 10 showed a significant increase in the GABA levels in their blood). From this, it can be seen that the subjects increase the GABA level in vivo by taking the P. acidilactici TCI550, to achieve various effects of calming and stabilizing nerves, improving neurological health, enhancing cognitive ability and the like.

Referring to FIG. 6. Before taking (i.e., week 0), the average BDNF level of the blood of 10 subjects was 4.93 ng/mL. After taking for 2 consecutive weeks (i.e., week 2), the average BDNF level of the blood of the 10 subjects was 5.66 ng/ml, which was increased by 14.8% compared to that at week 0. After taking for 4 consecutive weeks (i.e., week 4), the average BDNF level of the blood of the 10 subjects was 6.41 ng/mL, which was increased by 30% compared to that at week 0. In other words, taking the P. acidilactici TCI550 viable bacteria capsule for 4 consecutive weeks can increase the BDNF level in vivo. Moreover, more than half of the 10 subjects were improved, with the improvement rate reaching 80% (i.e., 8 out of 10 showed a significant increase in the BDNF level of their blood). From this, it can be seen that the subjects increase the BDNF level in vivo by taking the P. acidilactici TCI550, to achieve various effects of calming and stabilizing nerves, improving neurological health, enhancing cognitive ability, aiding in sleep, improving brain plasticity and the like.

### 7-2. Autonomic Nerve Detection

When the autonomic nerve was disordered, the sympathetic nerve will be in a state of excitement during sleep, so that the body and brain were unable to rest properly. Therefore, whether the autonomic nerve is healthy or disordered can be judged by measuring the autonomic nerve activity during sleep.

Detection method: 10 subjects used a LARGAN sleep detection system (LARGAN HEALTH) at home and sat quietly for 10 minutes before bedtime to measure their heart rate variability (HRV) data. Heart rate variability (HRV) is an important index for measuring autonomic nervous system activity. LF in the data is the overall activity of the sympathetic nerves, which represents the level of tension and excitement of the subjects.

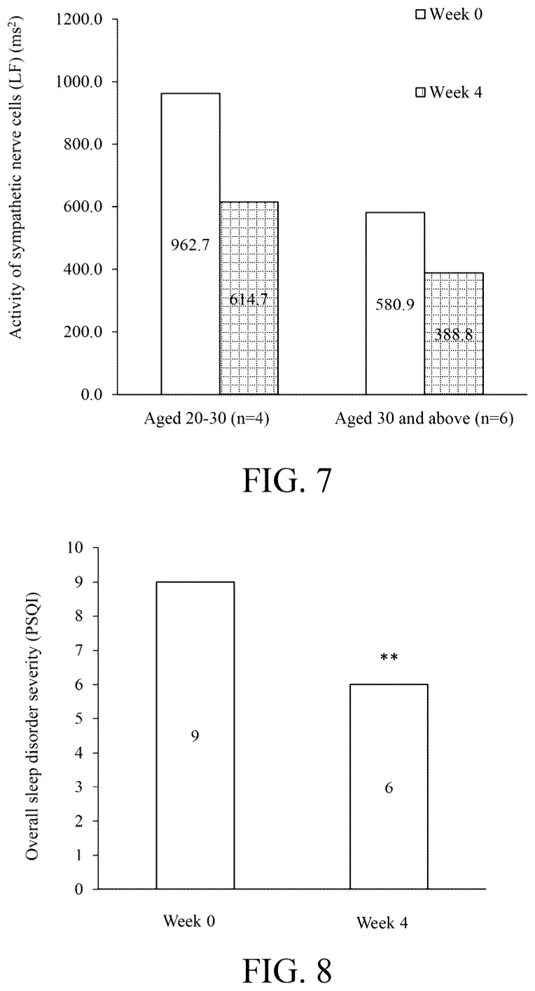

Moreover, the numerical values of heart rate variability will decrease with age, and gender has a smaller impact on the heart rate variability than age. In addition, gender differences will decrease when the age is above 30 years old. Therefore, aged 30 is used as the cut-off point for subsequent analysis. Out of the 10 subjects, there were 4 subjects aged from 20 to 30, and there were 6 subjects aged 30 and above. The average age of the 4 subjects was aged from 20 to 30 was 26 years old; and the average age of the 6 subjects aged 30 and above was 43 years old. The analysis results are as shown in FIG. 7.

Judgment criteria-LF reference value: 263.9 ms² to 896.7 ms² for individuals aged from 20 to 30; and 112.9 ms² to 543.2 ms² for individuals aged 30 and above.

Referring to FIG. 7. The average sympathetic nerve cell activity of the 4 subjects aged from 20 to 30 at week 0 was 962.7 ms², which was higher than a general LF reference value. Therefore, the 4 subjects had the possibility of anxiety and insomnia. After taking for 4 consecutive weeks (i.e., week 4), the average sympathetic nerve cell activity of the 4 subjects aged from 20 to 30 was 614.7 ms², which was decreased by 36.1% compared with that at week 0, which indicates that the neural state of the subject changed from tension and excitement to relax and restful sleep. The average sympathetic nerve cell activity of the 6 subjects aged 30 and above at week 0 was 580.9 ms², which was higher than a general LF reference value. Therefore, the 6 subjects had the possibility of anxiety and insomnia. After taking for 4 consecutive weeks (i.e., week 4), the average sympathetic nerve cell activity of the 6 subjects aged 30 and above was 388.8 ms², which was decreased by 3.1% compared with that at week 0, which indicates that the neural state of the subject changed from tension and excitement to relax and restful sleep. From this, it can be seen that the subjects soothe the nerves before bedtime by taking the P. acidilactici TCI550, thereby calming and stabilizing the nerves, improving neurological health, aiding in sleep, etc. Moreover, when sleep is sufficient, various effects of enhancing cognitive ability and improving brain plasticity can be even achieved.

### 7-3. Assessment of Sleep Disorder: Pittsburgh Sleep Questionnaire

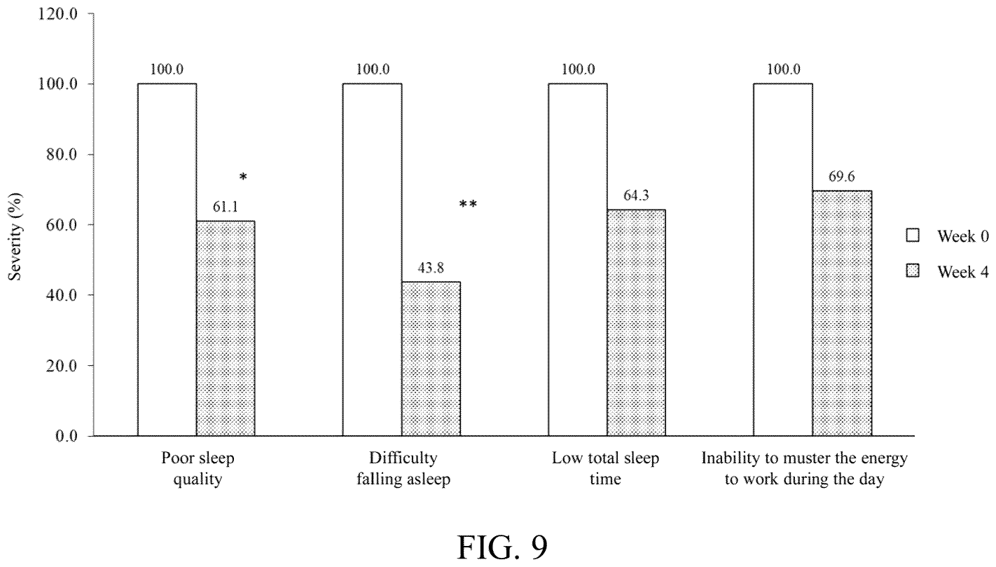

Detection method: The Pittsburgh Sleep Quality Index (PSQI): Western Ontario and McMaster Universities Osteoarthritis Index (WOMAC) is a score that assesses across seven dimensions: subjective sleep quality, sleep latency, total sleep time, sleep efficiency, sleep disorder, sleep medication use, daytime dysfunction. Regarding each dimension, the minimum score was 0, the maximum score was 3, and a total score range was 0 to 21. When the PSQI score was greater than 5, it indicates the sleep disorders, and the higher the score, the worse the sleep quality. The 10 subjects completed the Pittsburgh Sleep Quality Form at week 0 and week 4 to assess sleep quality before and after taking the P. acidilactici TCI550. The assessment results are as shown in FIG. 8 and FIG. 9.

Referring to FIG. 8. At week 0, the overall sleep disorder severity (PSQI) of the 10 subjects was 9 points, which indicates that the subjects suffered from the sleep disorder. After taking for 4 consecutive weeks (i.e., week 4), the overall sleep disorder severity (PSQI) of the 10 subjects decreased to 6 points, which still indicates that the subjects had the sleep disorder, but the decreased score also indicates that the sleep quality was improved. Out of the 10 subjects, the rate of the subjects who felt improvement was 80% (i.e., 8 subjects).

In addition to scoring the overall sleep disorder severity, 10 subjects were also assessed for the judgment items for the sleep disorder severity one by one: “poor sleep quality, difficulty falling asleep, low total sleep time and inability to muster the energy to work during the day”. All the scores at week 0 were regarded as 100%, and the relative percentage of the scores at week 4 was converted to infer the improvement level.

Referring to FIG. 9. At week 4, the severity of “poor sleep quality” decreased to 61.1%, the severity of “difficulty falling asleep” decreased to 43.8%, the severity of “low total sleep time” decreased to 64.3%, and the severity of “inability to muster the energy to work during the day” decreased to 69.6%. The scores for poor sleep quality and difficulty falling asleep were significantly reduced by 38.9% and 56.2%, respectively, and out of the 10 subjects, the rate of the subjects who felt improvement was 70% (i.e., 7 subjects).

From this, it can be seen that the subjects improve the sleep quality and alleviate the sleep disorder by taking the P. acidilactici TCI550, and the sleep quality was improved, which in turn maintains neurological health and achieves the effect of improving neurological health.

### 7-4. Assessment of Cognitive Disorder:

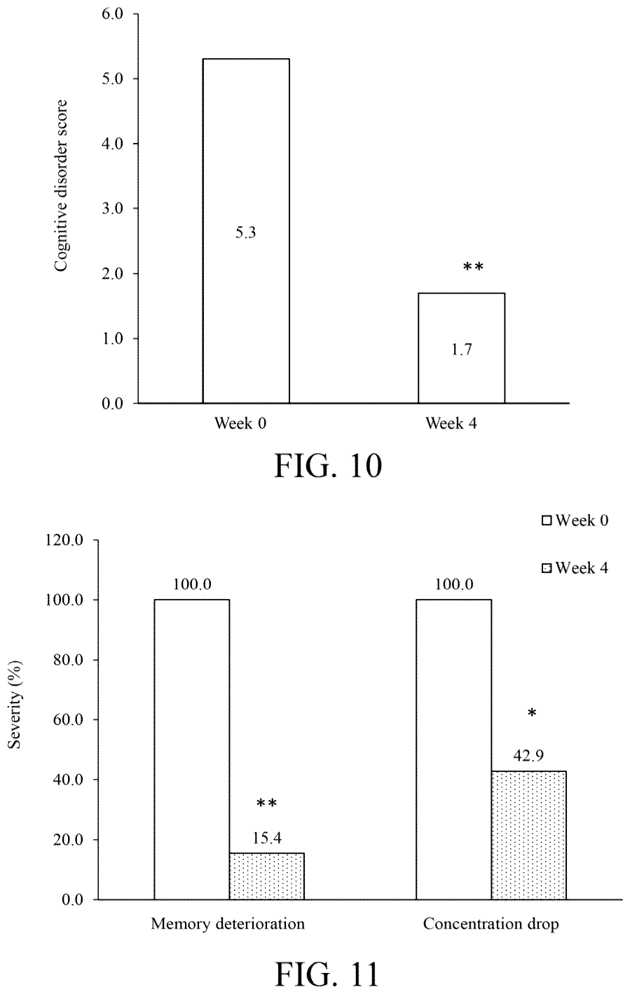

Detection method: The “Taiwan Cognitive Function Form” is a diagnostic tool for assessing whether the subjects have memory deterioration, are prone to indecision, and are unable to concentrate. The Taiwan Province Cognitive Function Form can assess the influence of the cognitive function on daily life, with a total of 5 questions. The higher the score, the worse the cognitive function. The 10 subjects completed the Taiwan Cognitive Function Form at week 0 and week 4 to assess the cognitive function before and after taking the P. acidilactici TCI550. The assessment results are as shown in FIG. 10 and FIG. 11.

Referring to FIG. 10. At week 0, the cognitive disorder scores of the 10 subjects were 5.3. After taking for 4 consecutive weeks (i.e., week 4), the cognitive disorder scores of the 10 subjects decreased to 1.7, which indicates that the cognitive disorder scores of the 10 subjects decreased significantly by 67.9%. Moreover, out of the 10 subjects, the rate of the subjects who felt improvement was 90% (i.e., 9 subjects).

In addition to the cognitive disorder scores, 10 subjects were also assessed for the judgment items for the sleep disorder severity: “memory deterioration and concentration drop”. All the scores at week 0 were regarded as 100%, and the relative percentage of the scores at week 4 was converted to infer the improvement level.

Referring to FIG. 11. At week 4, the severity of “memory deterioration” decreased to 15.4%, and the severity of “concentration drop” decreased to 42.9%. The 10 subjects felt improvement in memory and concentration, and the severities were reduced by 84.6% and 57.1%, respectively. Moreover, out of the 10 subjects, the rate of the subjects who felt improvement in memory was 80% (i.e., 8 subjects), and the rate of the subjects who felt improvement in concentration was 60% (i.e., 6 subjects).

From this, it can be seen that the subjects improve the memory and concentration, and improve the cognitive function, by taking the P. acidilactici TCI550.

### 7-5. Somatosensory Questionnaire:

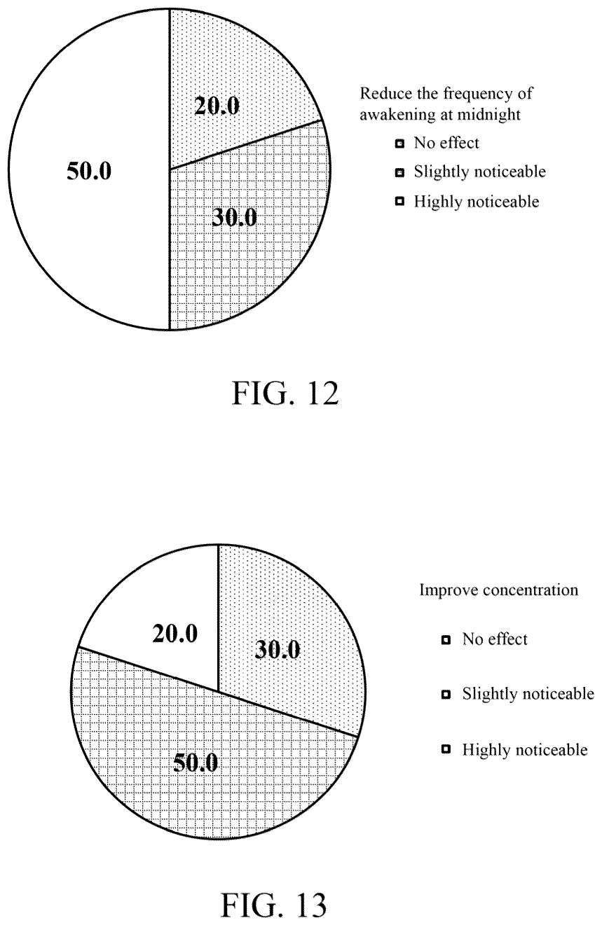

Detection method: The 10 subjects took the P. acidilactici TCI550 capsule for 4 weeks, and then completed the somatosensory questionnaire at week 4. The following three options are provided: “no effect”, “slightly noticeable”, and “highly noticeable” regarding whether the P. acidilactici TCI550 capsule “reduces the frequency of awakening at midnight” and “improves concentration”. The analysis of the questionnaire results is shown in FIG. 12 and FIG. 13.

Referring to FIG. 12. For the effect of the P. acidilactici TCI550 on reducing the frequency of awakening at midnight, 50% of the 10 subjects felt “highly noticeable”, 30% felt “slightly noticeable”, and 20% felt “no effect”. From this, it can be seen that 8 subjects felt improvement for “the P. acidilactici TCI550 bacteria reduces the frequency of awakening at midnight”.

Referring to FIG. 13. For the effect of the P. acidilactici TCI550 on improving concentration, 20% of the 10 subjects felt “highly noticeable”, 50% felt “slightly noticeable”, and 30% felt “no effect”. From this, it can be seen that 7 subjects felt improvement for “the P. acidilactici TCI550 bacteria improves concentration”.

From this, it can be seen that taking the P. acidilactici TCI550 for a long time effectively improve the sleep quality of the subject and improve the concentration of the subject.

To sum up, according to the P. acidilactici TCI550 deposited under the accession number of BCRC911167 in any embodiment of the present disclosure, it can be used for preparing a composition for improving neurological health, a composition for improving cognition enhancement, or a combination thereof. In other words, the aforementioned composition has one or more of the following functions: improving brain plasticity, calming nerves, reducing the overall activity of sympathetic nerve cells, improving responsiveness, concentration, memory, improving responsiveness, concentration, memory, improving the expression levels of the serotonin production-related genes (e.g., the TPH1 gene, the DDC gene or a combination thereof), and the melatonin synthesis-related genes (e.g., the ASMT gene, the AANAT gene or a combination thereof), the depression resistance-related genes (e.g., the SIRT1 gene, the BDNF gene or a combination thereof) in the nerve cells, increasing the level of the γ-aminobutyric acid in blood, increasing the level of the brain-derived neurotrophic factor in blood, etc.

Although the instant disclosure has been described in considerable detail with reference to certain preferred embodiments thereof, the disclosure is not for limiting the scope of the invention. Persons having ordinary skill in the art may make various modifications and changes without departing from the scope and spirit of the invention. Therefore, the scope of the appended claims should not be limited to the description of the preferred embodiments described above.

What is claimed is:

1. A method for improving neurological health, comprising administering to a subject in need thereof a composition comprising an effective dose of Pediococcus acidilactici and/or metabolites thereof, wherein the P. acidilactici is P. acidilactici TCI550 deposited under an accession number of DSM34536.

2. The method according to claim 1, wherein the improvement of neurological health is improving brain plasticity, calming nerves, or a combination thereof.

3. The method according to claim 1, wherein the composition has a capability to reduce overall activity of sympathetic nerve cells.

4. The method according to claim 1, wherein the composition has a capability to increase expression levels of serotonin production-related gene, melatonin synthesis-related gene, depression resistance-related gene, or any combination thereof in a nerve cell.

5. The method according to claim 4, wherein the serotonin production-related gene is tryptophan hydroxylase (TPH) gene, dopa decarboxylase (DDC) gene, or a combination thereof.

6. The method according to claim 4, wherein the melatonin synthesis-related gene is acetylserotonin O-methyltransferase (ASMT) gene, arylalkylamine acetyltransferase (AANAT) gene, or a combination thereof.

7. The method according to claim 4, wherein the depression resistance-related gene is sirtuin 1 (SIRT1) gene, brain-derived neurotrophic factor (BDNF) gene, or a combination thereof.

8. The method according to claim 1, wherein the composition has a capability to promote secretion of γ-aminobutyric acid (GABA) by nerve cells.

9. The method according to claim 1, wherein the P. acidilactici TCI550 has a capability to produce γ-aminobutyric acid and increase a level of the γ-aminobutyric acid in blood.

10. The method according to claim 1, wherein the P. acidilactici TCI550 has a capability to increase a level of brain-derived neurotrophic factor (BDNF) in blood.

11. The method according to claim 1, wherein the effective dose is 50 mg/day.

12. A method for improving cognition, comprising administering to a subject in need thereof a composition comprising an effective dose of P. acidilactici and/or metabolites thereof, wherein the P. acidilactici is P. acidilactici TCI550 deposited under an accession number of DSM34536.

13. The method according to claim 12, wherein the composition has a capability to improve responsiveness, concentration, memory, or any combination thereof.

14. The method according to claim 12, wherein the composition has a capability to increase expression levels of serotonin production-related gene, melatonin synthesis-related gene, depression resistance-related gene, or any combination thereof in a nerve cell.

15. The method according to claim 14, wherein the serotonin production-related gene is tryptophan hydroxylase (TPH) gene, dopa decarboxylase (DDC) gene, or a combination thereof.

16. The method according to claim 14, wherein the melatonin synthesis-related gene is acetylserotonin O-methyltransferase (ASMT) gene, arylalkylamine acetyltransferase (AANAT) gene, or a combination thereof.

17. The method according to claim 14, wherein the depression resistance-related gene is sirtuin 1 (SIRT1) gene, brain-derived neurotrophic factor (BDNF) gene, or a combination thereof.

18. The method according to claim 12, wherein the composition has a capability to promote secretion of γ-aminobutyric acid (GABA) by nerve cells.

19. The method according to claim 12, wherein the P. acidilactici TCI550 has a capability to produce γ-aminobutyric acid and increase a level of the γ-aminobutyric acid in blood.

20. The method according to claim 12, wherein the P. acidilactici TCI550 has a capability to increase a level of brain-derived neurotrophic factor (BDNF) in blood.

21. The method according to claim 12, wherein the effective dose is 50 mg/day.

22. A composition, comprising P. acidilactici and a food additive, wherein the P. acidilactici is P. acidilactici TC1550 deposited under an accession number of DSM34536, and the food additive is at least one of a group consisting of: soybean milk powder, trehalose, indigestible maltodextrin, and sorbitol.
